(19) United States
(12) Patent Application Publication
(10) Pub. No.: US 2010/0010062 A1
(43) Pub. Date: Jan. 14, 2010

(54) METHOD OF TREATMENT USING EPROSARTAN

(75) Inventors: Paulus A.J. Link, Bunnik (NL); Marcellus M. van der Hulst, Hoogland (NL); Gerhard-Wilhelm Bielenberg, Alfeld (DE); Cornelis R. van den Akker, Diemen (NL)

Correspondence Address: MAYER BROWN LLP, P.O. BOX 2828, CHICAGO, IL, 60690, US

(73) Assignee: Solvay Pharmaceuticals B.V., CP Weesp (NL)

(21) Appl. No.: 12/500,425

(22) Filed: Jul. 9, 2009

Related U.S. Application Data
(60) Provisional application No. 61/080,067, filed on Jul. 11, 2008.

(30) Foreign Application Priority Data
Jul. 11, 2008 (EP) 08 160211.2

(51) Int. Cl. A61K 31/4178; C07D 409/06

(52) U.S. Cl. 514/397; 548/315.1

(57) ABSTRACT

The disclosed invention relates to a method of treatment of a disorder modulated by blocking angiotensin II (AII) receptors, and particularly selected from the group consisting of hypertension, congestive heart failure, renal failure, and combinations thereof, by administering to a subject in need thereof an effective dose of an eprosartan compound. With reference to the Recommended Effective Daily Dose of 600 mg, calculated on the basis of eprosartan administered in the form of eprosartan mesylate, it has now been found that a lower dose of eprosartan can be administered when the eprosartan compound is eprosartan acid. This dose is in the range of from 410 to 490 mg, most preferably about 450 mg.

### FIELD OF THE INVENTION

The present invention relates to a method of treatment of a disorder modulated by blocking angiotensin II (AII) receptors, and particularly selected from the group consisting of hypertension, congestive heart failure, renal failure, and combinations thereof, by administering to a subject in need thereof, an eprosartan compound in a dose sufficient to treat such disorders (the Recommended Effective Daily Dose). The invention further relates to providing a drug product that is bioequivalent with a reference drug product that has eprosartan mesylate as the active substance. Also, the invention pertains to a pharmaceutical dosage unit for the administration of eprosartan and to a pharmaceutical formulation comprising eprosartan.

### BACKGROUND OF THE INVENTION

Eprosartan is (E)-α-[2-n-butyl-1-[(4-carboxy phenyl)methyl]-1H-imidazol-5-yl]methylene-2-thiophenepropionic acid. Eprosartan is the subject of U.S. Pat. No. 5,185,351 (the '351 patent), issued Feb. 9, 1993. See also EP 0 955 294, which has a similar disclosure. EP 0 955 294 describes the synthesis and general use of eprosartan and related compounds as All receptor antagonists. A synthesis example of eprosartan acid is included. The preference in this disclosure is given to the methyl sulfonate salt (eprosartan mesylate).

The state of the art with respect to the aforementioned method of treatment is the existing commercial form of eprosartan, namely eprosartan mesylate.

However, the variable and mean absolute bioavailability of eprosartan mesylate is approximately 13%. As a result, high doses may be required for an effective treatment of hypertension, congestive heart failure and renal failure. Effective daily doses range from 400 to 800 mg, calculated on the basis of eprosartan.

The Recommended Effective Daily Dose of eprosartan mesylate is 600 mg, calculated on the basis of eprosartan. In some cases, a starting regimen is prescribed in which an initial dose of 300 mg per day is administered. The Recommended Effective Daily Dose can be achieved by the once daily administration of the aforementioned eprosartan mesylate 600 mg tablets or by the twice daily administration of eprosartan mesylate 300 mg tablets.

The Recommended Effective Daily Dose can also be characterized with reference to the plasma levels of eprosartan achieved. For example, following the administration of the eprosartan compound to a human subject at the Recommended Effective Daily Dose, the subject exhibits at least one of:

(a) a mean plasma Cₘₐₓ of eprosartan of between 2200 and 3600 ng/ml; or
(b) a mean plasma AUC_{0-T} of eprosartan of between 8000 and 11000 hr·ng/ml.

By way of further background, the following documents reference eprosartan.

U.S. Pat. No. 5,656,650 relates to pharmaceutical compositions comprising, in addition to an All receptor antagonist, a second agent being a diuretic, a calcium channel blocker, a β-adrenoceptor blocker, a renin inhibitor, or an angiotensin converting enzyme inhibitor. As to eprosartan, the document exemplifies oral dosage forms of 100 mg eprosartan acid. The total daily dose of the All receptor antagonist is broadly indicated to be from about 5 mg to about 1000 mg. The document does not suggest any change to the aforementioned Recommended Effective Daily Dose.

WO 99/25321 addresses the high tablet weight of eprosartan mesylate dosage units. It is proposed to provide high drug load tablets on the basis of anhydrous eprosartan acid. The document refers to the above-mentioned typical effective dose, viz., 600 mg (calculated based on eprosartan). The document refers to 600 mg dosage units and does not indicate any alteration of the aforementioned Recommended Effective Daily Dose. The reference apparently does not fully succeed in providing the required 600 mg dosage units. Although a broad dosage range of 50 mg to 1 g is given, the preferred dosage units contain from about 200 mg to about 400 mg of the eprosartan acid. It is indicated that these are to be taken 1-4 times daily, preferably 1-2 times daily. This is commensurate with the aforementioned state of the art doses of 300 mg, or 600 mg total.

FR 2 886 150 addresses the administration of active substances whose solubility varies strongly with varying gastric pH. Eprosartan is given as an example. The problem is that these drugs, within the same patient, may lead to variable plasma levels depending, e.g., on the time of day the drug is taken and whether the patient has a fed or empty stomach. The reference proposes to address this problem by providing the active substance with a coating or by combining it with a matrix, which allows the controlled release of the active substance. As an example, a 300 mg dosage unit of eprosartan is given. The reference does not indicate any alteration of the Recommended Effective Daily Dose, i.e., considering the standard dosage amounts in the art. This exemplified dosage unit would normally be given twice daily so as to reach the standard effective dose of 600 mg eprosartan.

FR 2 882 260 refers to multiple daily administrations of AII receptor antagonists, on account of their low half-life values. As a solution, it is proposed to provide multi-microparticulate dosage forms for the prolonged release of these compounds. For eprosartan, an example of a 400 mg microparticulate dosage unit is provided, which is indicated to prolong the duration of release by 6 hours. The document neither indicates the daily doses required, nor does it indicate any alteration of the Recommended Effective Daily Dose.

With respect to the latter two references, it is noted that in the present invention, it is particularly desired to provide a straightforward immediate-release formulation for eprosartan.

“Immediate Release Formulation” means any formulation such that by the time eprosartan leaves the stomach, it is either in solution or in the form of a suspension of fine particles, i.e., a form from which eprosartan can be readily absorbed. More particularly, the term “Immediate Release” refers to a release of at least about 75%, and preferably at least about 90% of the drug in a dissolved form from the dosage form within about 90 minutes, preferably within about 60 minutes. Most particularly, the term “Immediate Release” refers to a release of at least about 75% within about 45 minutes. Preferred Immediate Release Formulations release at least about 90% of the drug in about 30 minutes, more preferably in about 15 minutes and most preferably at least about 95% of the drug in about 15 minutes. The release rates referred to are those as determined in accordance with the United States Pharmacopeia (USP) as described in Example 8.

None of the foregoing references addresses the desire to provide an eprosartan dosage unit on the basis of an eprosartan drug substance that has a better bioavailability than the standard in the art, viz., eprosartan mesylate.

### SUMMARY OF THE INVENTION

While the present invention is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. The various embodiments disclosed herein may be combined with other embodiments for the creation and description of yet additional embodiments.

It would be desired to provide an eprosartan compound having a better bioavailability than the current standard, eprosartan mesylate. This would allow achieving the Recommended Effective Daily Dose on the basis of a lower amount of drug or, in other words, providing bioequivalent tablets, having a lower amount of drug. This is generally recognized as a benefit to patients.

Surprisingly, it was found that eprosartan acid is a better bioavailable eprosartan compound.

Thus, one embodiment is a treatment with eprosartan as above, i.e., within the Recommended Effective Daily Dose, wherein the eprosartan compound is an effective dose of eprosartan acid.

In another embodiment, the dosage unit of eprosartan acid is about 410 to about 490 mg, preferably about 440 to about 460 mg, and particularly of about 450 mg. In a further embodiment, the eprosartan acid dosage unit is used in a treatment as referred to above, wherein the treatment comprises the once daily administration of said dosage unit. In still another embodiment, a pharmaceutical formulation comprises eprosartan acid and at least one excipient selected from the group consisting of: alpha lactose monohydrate and polyalcohols, such as mannitol.

Another embodiment is the use of eprosartan acid for the purpose of providing a drug product that is bioequivalent with a reference drug product comprising eprosartan mesylate as the active substance, wherein the bioequivalent dose of eprosartan acid is lower than the reference dose of eprosartan mesylate, calculated on the basis of eprosartan acid.

A further embodiment relates to formulations of eprosartan having improved bioavailability compared with the present marketed eprosartan mesylate formulations, processes for manufacturing these formulations, and methods of using the eprosartan formulations of the present invention in the treatment of certain disease states in mammals, in particular man.

### DESCRIPTION OF THE FIGURES

FIG. 1 depicts the XRPD pattern of polymorphic form cl eprosartan acid

FIG. 2 depicts the XRPD pattern of polymorphic form P of eprosartan acid

FIG. 3 is a graph representing Geometric Mean Eprosartan Plasma Concentration Time Profiles for all Treatments discussed in Example 8 (0-10 hour profiles)

### DETAILED DESCRIPTION OF THE INVENTION

### 1. Effective Dose in the Treatment of AII Receptor-Modulated Disorders

Eprosartan serves in the treatment of disorders modulated by blocking angiotensin II (AII) receptors. The invention particularly pertains to disorders selected from the group consisting of: hypertension, congestive heart failure, renal failure, and combinations thereof.

The method of treatment is by administering to a subject in need thereof, a dose of an eprosartan compound within the Recommended Effective Daily Dose, i.e., the level of administration of eprosartan commensurate with the doses approved for the treatment of the aforementioned disorders.

As mentioned above, the Recommended Effective Daily Dose is 600 mg (calculated based on eprosartan) as present in eprosartan mesylate.

The Recommended Effective Daily Dose serves to introduce into the subject's plasma a level of eprosartan corresponding to the level introduced upon the administration of crystalline eprosartan mesylate in a dosage amount of 600 mg.

More particularly, the Recommended Effective Daily Dose is a dose of an eprosartan compound following its administration to human subjects, and the subjects exhibit at least one of:
(a) A mean plasma Cₘₐₓ of eprosartan of between 2200 and 3600 ng/ml; or
(b) A mean plasma AUC₀₋ₜ of eprosartan of between 8000 and 11000 hr·ng/ml; “AUC” means the Area Under plasma concentration time Curve.

In one embodiment, the eprosartan acid is administered in an effective daily dose of from about 410 to about 490 mg. Preferably, eprosartan acid is administered in a daily dose of from about 420 to about 480 mg. More preferably, said daily dose ranges from about 430 to about 470 mg, and even more preferably from about 440 to about 460 mg. Most preferably, eprosartan is provided in a daily dose of about 450 mg. Further, the amount of eprosartan acid is selected from the group consisting of: about 410 mg, about 420 mg, about 430 mg, about 440 mg, about 450 mg, about 460 mg, about 470 mg, about 480 mg, and about 490 mg.

Another embodiment pertains to dosage units comprising eprosartan acid in a single daily dose within any of the foregoing ranges, and most preferably comprising 450 mg of eprosartan acid.

Whilst the benefit of a single administration of an eprosartan acid dosage unit within the above ranges is more fully enjoyed in comparison with a single 600 mg eprosartan mesylate dosage unit, it will be understood that it will be possible to administer the daily dose of eprosartan acid as mentioned above, by multiple administrations of a lower dose of eprosartan acid. Upon such a multiple administration, it is to be taken into account that the total daily dose of eprosartan acid will be about 410 to about 490 mg, preferably about 420 to about 480 mg, more preferably about 430 to about 470 mg, still more preferably about 440 to about 460 mg and, most preferably about 450 mg.

If the eprosartan acid is administered in multiple dosage amounts during a day, this is preferably in two half-dosage amounts of the daily dose. The half dosage amounts can be provided for by an appropriate score in a tablet having the daily dosage amount or it can be provided for by means of separate dosage units comprising the appropriate half dosage strength. If desired, such half-strength dosage units can also be administered simultaneously so as to have the effect of a single daily dose.

The purpose of the invention is to provide a treatment that can be considered similar to the treatment with eprosartan mesylate. By “similar,” it is meant that the treatment with an eprosartan compound at a dosage level is considered bioequivalent with the appropriate dosage level of eprosartan mesylate.

Surprisingly, it was found that if the eprosartan compound is eprosartan acid, the dosage level of the eprosartan acid that is bioequivalent with a dosage level of x mg eprosartan mesylate (with x calculated on eprosartan acid and being between 400 and 800 mg), is lower than x mg.

Another embodiment would allow a pro rata lower amount of eprosartan acid in cases where other dosage amounts of the marketed drug eprosartan mesylate might be employed. Generally, the dosage level (calculated on eprosartan acid) achievable with the invention herein is from 70% to 80%, and particularly 75%, of the effective dose (calculated on the basis of eprosartan acid) of eprosartan mesylate. Thus, e.g., in the event of the initial treatment dose of 300 mg eprosartan mesylate as referred to above, the present invention allows the administration of 210-240 mg eprosartan acid, and preferably 225 mg. Similar conversions will hold for other dosage amount of eprosartan mesylate.

The substantial reduction of the eprosartan strength in the treatment of the invention is unforeseen in the art. Therein, the problem of finding a better bioavailable, bioequivalent alternative to eprosartan mesylate is not addressed. References on eprosartan acid, and its salts, include a variety of patent documents from the early 1990s in which formulation examples are included, e.g., of eprosartan acid (75 mg or 100 mg): EP 0 955 294, WO 92/10189, U.S. Pat. No. 5,418,250, U.S. Pat. No. 5,185,351, WO 199/10097, U.S. Pat. No. 5,656,650, WO 92/10181, U.S. Pat. No. 6,034,114, WO 92/10182, U.S. Pat. No. 6,028,091, EP 561 977, U.S. Pat. No. 6,025,380, EP 561 876. These examples are not related to a method of treatment using eprosartan at the aforementioned effective doses.

With reference to the aforementioned dose ranges of eprosartan acid, which surprisingly serve to administer eprosartan at a level within the Recommended Effective Daily Dose, further embodiments also includes the pharmaceutical formulations per se that can be used as described above. Thus, one embodiment is a pharmaceutical formulation comprising about 410 to about 490 mg, preferably about 420 to about 480 mg, more preferably about 430 to about 470 mg, even more preferably about 440 to about 460 mg, and most preferably about 450 mg of eprosartan acid. The amount of eprosartan acid is selected from the group consisting of: about 410 mg, about 420 mg, about 430 mg, about 440 mg, about 450 mg, about 460 mg, about 470 mg, about 480 mg, and about 490 mg.

### 2. Eprosartan Drug Product for Use in the Treatment of AII Receptor-Modulated Disorders

One embodiment pertains to an eprosartan drug product for use in a method of treating a disorder modulated by blocking angiotensin II (AII) receptors, and particularly selected from the group consisting of hypertension, congestive heart failure, renal failure, and combinations thereof, by administering to a subject in need thereof, an effective dose of an eprosartan compound at a level of administration so as to achieve the Recommended Effective Daily Dose, wherein the eprosartan compound in the drug product is eprosartan acid.

As to the effective dose of eprosartan acid, the considerations given above under (1.) are applicable as well.

### 3. Providing a Bio-Equivalent Eprosartan Drug Product

By virtue of this invention, it has become possible, and unexpectedly so, to provide a drug product that is bioequivalent with the existing eprosartan mesylate drug product, yet on the basis of a lower dosage strength of eprosartan.

One embodiment resides in the use of eprosartan acid for the purpose of providing a drug product that is bioequivalent with a reference drug product comprising crystalline eprosartan mesylate as the active substance, wherein the bioequivalent dose of eprosartan acid is lower than the reference dose of eprosartan mesylate, calculated on the basis of eprosartan acid.

Similarly, an additional embodiment resides in a method of providing an eprosartan drug compound that is bioequivalent with a reference drug product comprising eprosartan mesylate, by providing the eprosartan drug compound and registering it with a regional or national authority responsible for the grant of a Marketing Authorization for drugs (such as the US FDA (United States Food and Drug Administration)) on the basis of the appropriate bioequivalency studies, wherein the eprosartan drug compound is eprosartan acid. Here too, the bioequivalent dose of eprosartan acid is lower than the reference dose of eprosartan mesylate, calculated on the basis of eprosartan acid. The concept of bioequivalency is known in the art.

In the framework of the present invention, the expression bioequivalent means: the absence of a significant difference in the rate and extent to which the eprosartan becomes available after administration of the new formulation and the 600 mg mesylate formulation in plasma under similar conditions in an appropriately designed study. The absence of a “significant difference” means that the 90% confidence interval for the AUC ratio and the 90% confidence interval for the Cₘₐₓ ratio are lying within an acceptance interval of 80-125%, preferably in an acceptance interval of 90-111% and most preferably in an acceptance interval of 95-105%. Appropriate designs for bioequivalence studies are well known to the skilled person.

This concept of bioequivalency serves to avoid an undue repetition of human clinical trials, by allowing a drug manufacturer (usually indicated as a “generic drug manufacturer”) to register a copy of a reference drug on the basis of limited trials, in which, it is merely shown that the copy is bioequivalent with the reference drug (the inference being that a bioequivalent drug by definition will have similar efficacy and safety).

In terms of providing a drug that is bioequivalent with eprosartan mesylate, the effective daily dose of eprosartan acid will be lower than the Recommended Effective Daily Dose, calculated on the basis of eprosartan acid, as present in eprosartan mesylate, the reference drug.

Without wishing to be bound by theory, it is believed that a further advantage can be attributed to the relatively high bioavailability of eprosartan acid, viz. a lower variability of pharmacokinetic parameters. Thus, the selection of eprosartan acid as the active ingredient will lead to better possibilities of providing a bioequivalent product with a lower sensitivity for small dosage or formulation changes.

### 4. Eprosartan Acid

(E)-α-[2-n-Butyl-1-[(4-carboxyphenyl)methyl]-1H-imidazol-5-yl]methylene-2-thio-phenepropionic acid (further referred to as eprosartan acid) is known to exist in a crystalline form from U.S. Pat. No. 5,185,351. Reference should be made to U.S. Pat. No. 5,185,351 for its full disclosure, the entire disclosure of which is incorporated herein by reference. Eprosartan acid is an amphiphilic molecule containing two acidic (allylic carboxylic acid and phenylic carboxylic acid) and one basic (imidazole) functional groups. At lower pH (below 2) the imidazole nitrogen will be protonated (form ii). As the pH increases, the allylic carboxylic group will be deprotonated (form iii). Estimated pKa of the allylic carboxylic group is 2.9. As the pH increases further, the phenylic carboxylic group will be deprotonated (form iv) followed by the deprotonation of the protonated imidazole group (form v). The estimated pKa of the phenylic carboxylic group is 5.9 and that of imidazole group is 6.8.

In accordance with the invention, the eprosartan acid is not restricted to any particular form, e.g., crystalline or amorphous. In one embodiment, the eprosartan acid is crystalline.

Crystalline eprosartan acid exists in two different polymorphic forms—the α (alpha) and β (beta) forms. The α polymorphic form of eprosartan acid has the highest thermodynamic stability of up to 200° C. Although the Examples use the α form of eprosartan acid, the β form of eprosartan acid can also be used in the same way.

The α polymorphic form of eprosartan acid is prepared by crystallizing the compound from acid medium (e.g. from acetic acid or formic acid) or by crystallizing the compound from ethanol/water while seeding. This polymorphic form is also described in WO 99/25321 and U.S. Pat. No. 5,185,351, although these publications are silent as to the way of preparation. The β polymorphic form of eprosartan acid is prepared by crystallizing the compound from ethanol/water without seeding.

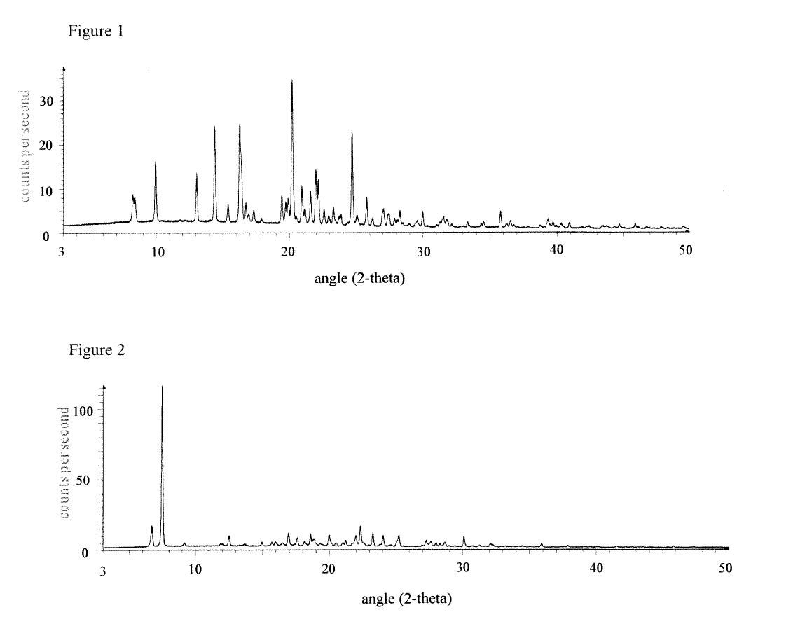

The anhydrous forms of eprosartan acid β polymorphic form exhibits a melting range, expressed as onset value in the DSC curve, of 264-269° C. The anhydrous forms of eprosartan acid α polymorphic form exhibits a single thermal event, a melting endotherm at about 269° C. No significant weight loss prior to melting is observed in its TGA (thermo gravimetric analysis), for both compounds, suggesting that these compounds do not contain significant quantities of surface adsorbed water and/or residual solvents. The powder X-ray diffraction (XRD) patterns of eprosartan acid α polymorphic form is presented in FIG. 1. The powder X-ray diffraction (XRD) patterns of eprosartan acid β polymorphic form is presented in FIG. 2.

### 5. Formulations

One embodiment of the present invention provides a formulation comprising eprosartan acid containing an amount of active ingredient that is considerably lower than a comparable formulation containing eprosartan mesylate, while maintaining the same bioavailability of the active ingredient. A further embodiment provides low dose eprosartan formulation comprising an amount of eprosartan acid, which is between about 68.3% and about 81.7%, particularly between about 70% and about 80%, and more particularly about 75%, of the calculated amount of eprosartan acid present in the comparable formulation comprising eprosartan mesylate. This means, e.g., that an eprosartan formulation comprising more than 420 mg and less than or equal to 480 mg crystalline eprosartan acid, and preferably about 450 mg, is bioequivalent to a comparable formulation containing 600 mg eprosartan acid in the form of eprosartan mesylate. This finding is surprising and unknown in the art. This also means that an eprosartan formulation comprising more than 210 mg and less than or equal to 240 mg crystalline eprosartan acid, and preferably about 225 mg, is bioequivalent to a comparable formulation containing 300 mg eprosartan acid in the form of eprosartan mesylate.

A comparable formulation means a formulation having the same release characteristics, but for the amount of the active ingredient, i.e., as is known to the skilled person. The rate and amount of release of an active compound from a pharmaceutical formulation can be influenced by the composition of excipients (i.e., the non-active constituents) of the formulation.

In another embodiment, a pharmaceutical composition is provided comprising an amount of eprosartan acid which is between about 68.3% and about 81.7%, particularly between about 70% and about 80%, and more particularly about 75%, of the calculated amount of eprosartan acid present in the comparable formulation comprising eprosartan mesylate, wherein following administration of both formulations to human subjects, the subjects exhibit at least one of:
(a) A mean plasma Cₘₐₓ ratio between 0.8-1.25 when comparing the eprosartan acid formulation with a comparable eprosartan mesylate formulation; or
(b) A mean plasma AUC₀₋ₜ ratio between 0.8-1.25 when comparing the eprosartan acid formulation with a comparable eprosartan mesylate formulation.

The indicated plasma Cₘₐₓ and plasma AUC₀₋ₜ ratios are preferably between 0.9-1.11 and even more preferably between 0.95-1.05. In a further embodiment, the amount of eprosartan acid is between about 68.3% and about 81.7% of the calculated amount of eprosartan acid present in the comparable formulation comprising eprosartan mesylate, while maintaining the plasma Cₘₐₓ ratio and/or plasma AUC₀₋ₜ ratio indicated above. In yet a further embodiment, the amount of eprosartan acid is between about 70% and about 80% of the calculated amount of eprosartan acid present in the comparable formulation comprising eprosartan mesylate, while maintaining the plasma Cₘₐₓ ratio and/or plasma AUC₀₋ₜ ratio indicated above. In still a further embodiment, the amount of eprosartan acid is about 75% of the calculated amount of eprosartan acid present in the comparable formulation comprising eprosartan mesylate, while maintaining the plasma Cₘₐₓ ratio and/or plasma AUC₀₋ₜ ratio indicated above. This means 225 mg eprosartan acid is comparable with a formulation containing 300 mg eprosartan mesylate, and 450 mg eprosartan acid is comparable with a formulation containing 600 mg eprosartan mesylate.

In a further embodiment, a pharmaceutical formulation is provided comprising about 410 to about 490 mg, preferably about 420 to about 480 mg, more preferably about 440 to about 460 mg, and most preferably about 450 mg of eprosartan acid and at least one pharmaceutically acceptable excipient, wherein following administration of the composition to a human subject, the subject exhibits at least one of:
(a) A mean plasma Cₘₐₓ of eprosartan of at least about 2200 ng/ml; or
(b) A mean plasma AUC₀₋ₜ, of eprosartan of at least about 8000 hr·ng/ml.

Another embodiment provides oral solid dosage forms of eprosartan acid for the treatment of diseases in which blockade of AII receptors is indicated, for example, in the treatment of hypertension, congestive heart failure and renal failure.

The formulations containing eprosartan acid exhibit significantly higher solubilities and faster dissolution in water, as well as in gastrointestinal fluids, than the comparable formulation containing eprosartan mesylate. The relative bioavailability in dogs for the eprosartan acid (α polymorphic form) formulation (which formulation is comparable in the composition and amount of auxiliary substances) is 61% higher and the mean AUC_{0→t} and Cₘₐₓ values increased 11% and 23%, respectively, when compared to the commercial eprosartan mesylate formulation. Consequently, lower strength tablets are needed for effective treatment of hypertension, congestive heart failure and renal failure, resulting in lower cost of goods and hence, significantly improved patient compliance.

A further embodiment relates to a low dose eprosartan formulation containing about 410 to about 490 mg, preferably about 420 to about 480 mg, more preferably about 430 to about 470 mg, even more preferably about 440 to about 460 mg, and most preferably about 450 mg of eprosartan acid, being bioequivalent to the same formulation containing 735 mg crystalline eprosartan mesylate. The eprosartan acid in the abovementioned formulation is preferably in the crystalline form and the amount of eprosartan in the formulation is preferably higher than 30% w/w and lower than 70% w/w. Further, the formulation does not contain more than 5% w/w of arginine and preferably no arginine at all. With respect to bioequivalence with the above-discussed 600 mg eprosartan formulation (735 mg of eprosartan mesylate), in a preferred embodiment, the daily dose applied in the invention is an amount of eprosartan acid of 420 mg to 450 mg, preferably comprised in a single daily dosage unit.

In another embodiment, it has been found that stable tablet formulations containing (E)-α-[2-n-butyl-1-[(4-carboxyphenyl)methyl]-1H-imidazol-5-yl]methylene-2-thiophenepropionic acid can be produced by pharmaceutical processes known in the field, e.g., wet-granulating, direct compression, spray drying, slugging, etc.

In a further embodiment, the formulation of the present invention comprises (E)α-[2-n-butyl-1-[(4-carboxyphenyl)methyl]-1H-imidazol-5-yl]methylene-2-thiophenepropionic acid and an alkali system in an amount greater than about 20% w/w of the composition, preferably comprising a mixture of at least two alkaline compounds in the ratio 1:20 to 20:1, and optionally one or more pharmaceutically acceptable excipients.

In a further preferred embodiment, the alkali system comprises a mixture of sodium bicarbonate and sodium carbonate, such as Buffered Soda™ (mixture of 41.5%-44.5% w/w sodium carbonate and 58.5%-55.5% sodium bicarbonate) and Effer-Soda™-12 (mixture of 83-90% w/w sodium bicarbonate and 10-17% w/w sodium carbonate) marketed by SPI Pharma. Effer-Soda™-12 is a highly stable, surface modified sodium bicarbonate powder. It is produced by converting the surface of sodium bicarbonate particles to sodium carbonate. Primarily, Effer-Soda™-12 contains 83-90% w/w sodium bicarbonate and 10-17% w/w sodium carbonate. The outer layer of sodium carbonate absorbs moisture (from the atmosphere or composition) and forms sodium sesquicarbonate, which is stable up to 70° C. temperature. This protection mechanism provided by the heat stable sodium sesquicarbonate prevents early effervescent reaction at ambient and elevated storage temperature conditions.

Surprisingly, it has been found that using an alkaline compound in the formulation, e.g., Effer-Soda™-12, alone or in a mixture, increases the bioavailability of eprosartan acid compared with eprosartan acid in the existing commercial formulation, as evidenced during an in vivo dog study. The mean relative bioavailability in dogs (n=6) for the eprosartan acid (α polymorphic form) formulation containing Effer-Soda™-12 is 107% higher and the mean AUC_{0→t} and Cₘₐₓ values increased 31% and 30%, respectively, when compared to the commercial eprosartan mesylate formulation. Consequently, lower strength tablets are needed for effective treatment of hypertension, congestive heart failure and renal failure, resulting in lower cost of goods and hence, significantly improved patient compliance.

In yet another embodiment, for ease of providing an eprosartan acid dosage unit that is bioequivalent with eprosartan mesylate, particularly in the Recommended Effective Daily Dose, the formulation is selected so as to provide a plasma concentration time curve of similar shape to that of the reference eprosartan mesylate formulation. To this end, the eprosartan acid is preferably formulated with lactose as an excipient, more preferably lactose monohydrate 200M. Such a preferred lactose grade is marketed, e.g., as Pharmatose® 200M. Other suitable excipients include other grades of alpha lactose monohydrate, dextrose, fructose, sucrose, cellulose, and polyalcohols. Polyalcohols, preferably spray-dried polyalcohols, include mannitol, sorbitol, and xylitol.

In connection herewith, a further embodiment pertains to a pharmaceutical formulation comprising the aforementioned effective daily dose of eprosartan and at least one excipient selected from the group consisting of: alpha lactose monohydrate and polyalcohols, more preferably lactose monohydrate 200M. Preferred formulations further comprise crospovidone (cross-linked N-vinyl-2-pyrrolidone) as a disintegrant. A still more preferred formulation comprises granules of lactose monohydrate 200M and microcrystalline cellulose, more preferably silicified microcrystalline cellulose as a binder, starch and crospovidone. As extragranular components, crospovidone is also present, as well as, magnesium stearate (lubricant).

A most preferred dosage unit comprises: 450 mg of eprosartan acid, 71.25 mg of alpha lactose monohydrate 200M (particularly Pharmatose® 200M), 60.0 mg of silicified microcrystalline cellulose (particularly Prosolv® SMCC90), 9.040 mg of starch, and 7.5 mg of crospovidone (particularly Polyplasdone® XL10). Preferably, the extragranular components are 7.5 mg of crospovidone (which brings the total of crospovidone in the dosage unit to 15 mg) and 7.5 mg of magnesium stearate.

One embodiment may comprise a diuretic compound as a further active ingredient, such as hydrochlorothiazide or furosemide. Preferably, the diuretic compound is hydrochlorothiazide. The amount of the diuretic present in a dosage unit is from about 1 mg to about 500 mg, preferably between about 10 and about 200 mg. The most preferred dose for hydrochlorothiazide is 12.5 mg. Naturally, these dose ranges can be adjusted on a unit basis as necessary to permit divided daily dose and, as noted above, the dose will vary depending on the nature and severity of the disease, weight of patient, special diets, and other factors.

### 6. Release

In one embodiment, the formulation may be produced as an immediate release or modified (sustained or targeted) release oral solid dosage form (capsule or tablet). As used herein, sustained release means any formulation that achieves slow release of the drug over an extended period of time. An example of a sustained release system is a matrix formulation. By targeted release is meant any formulation having an enteric coat or a sustained release coat where timed release is achieved by a barrier coating. As used herein, granulation means a solid containing the drug substance mixed with pharmaceutically acceptable carriers or excipients.

As mentioned above, the formulation pertains to dosage forms of the immediate release type. This refers to a release of at least about 75%, and preferably at least about 90% of the drug in a dissolved form from the dosage form within about 90 minutes, preferably within about 60 minutes. Preferred immediate release formulations release at least about 90% of the drug in about 30 minutes, more preferably in about 15 minutes and most preferably at least about 95% of the drug in about 15 minutes. The release rates referred to are those as determined in accordance with the United States Pharmacopeia (USP).

In a particularly preferred embodiment, the eprosartan formulation used is one exhibiting a release profile of: at least about 36% in about 5 minutes, at least about 95% in about 15 minutes, and 100% in about 30 minutes.

The release is determined in accordance with USP. In particular, this refers to dissolution testing using the USP Dissolution Apparatus II with a dissolution medium 0.2 M phosphate buffer at pH 7.5, a medium volume of 1000 ml with a temperature of 37±0.5° C. at a paddle speed of 50 rpm, taking samples with a sample volume of 10 ml and measuring in a QS Flow cell with a path length of 1 mm at a wavelength of 235 mm. The 0.2 M phosphate buffer was prepared by dissolving 302.6 g of disodium hydrogen phosphate dihydrate and 40.8 g of potassium dihydrogen phosphate in 10 litres of pure water. The pH was adjusted to 7.50±0.05 with addition of either 5 M sodium hydroxide or 85% phosphoric acid.

### 7. Manufacture

Eprosartan acid can be manufactured in accordance with the aforementioned '351 patent. It is preferably purified by recrystallization.

Pharmaceutical formulations of eprosartan acid can be manufactured generally in accordance with techniques known in the art. Preferably, the formulations are made in a wet-granulation process comprising dry mixing the active ingredient and excipients, adding water, and executing one or more granulation steps, e.g., in a fluid bed granulator, and processing the resulting granules into a dosage form, such as filling into capsules or compressing into tablets. Carriers or excipients may include, e.g., diluents, binders, and disintegrants. Further, particularly before tableting, a lubricant, such as magnesium stearate, can be added. Preferred diluents are lactose, microcrystalline cellulose, starch; the latter can also serve as a disintegrant. The carriers or excipients commonly used in pharmaceutical industry are well described in the literature, e.g., the Handbook of Pharmaceutical Excipients, A. Wade and P. J. Weller (Editors), American Pharmaceutical Association (1994). The dosage forms can be capsules or tablets for immediate release. The formulations can also be processed into matrix-based or film-coated dosage forms (beads, pellets or tablets) intended for modified or targeted release.

Any combination of pharmaceutically acceptable carriers or excipients, e.g., diluents, fillers, binders and disintegrants, in desired proportions, may be utilized with the spray dried or fluid bed granulated drug substance and immediate or modified release dosage forms of the present invention.

Pharmaceutically acceptable crystallization inhibitors include poly (vinyl pyrrolidone) and urea. Fillers and diluents include, but are not limited to, the following: lactose (hydrous as well as anhydrous), starch (unmodified (corn starch) or modified (for example, Starch 1500 available from Colorcon)), mannitol, sorbitol, cellulose, inorganic sulfates and phosphates. Disintegrants include, but are not limited to, the following: sodium starch glycolate, sodium carmellose and crosslinked polyvinyl pyrrolidone, and binders include, but are not limited to, the following: gelatin, corn starch, modified starch (Starch 1551, pregelatinized starch), hydroxypropyl methyl cellulose (HPMC), hydroxypropyl cellulose (HPC), sodium carboxy methyl cellulose, alginic acid, acacia and amino acids, such as glycine, L-arginine, etc. Examples of excipients suitable for modified release applications include, but are not limited to, the following: high molecular weight HPMCs, polymethacrylate polymers known as Eudragit, polyethylene oxide, Polyox® (Union Carbide Corporation), modified ethyl cellulose, Surelease® (Colorcon), crosslinked acrylic acid polymers, Carbopol® (BF Goodrich Speciality Chemicals) and waxy materials, such as glyceryl behenate (Compritol®), glyceryl palmitostearate (Precirol®), and Gelucires® [all from Gattefosse s.a., France] and carnauba wax.

Preferably, the pharmaceutically acceptable excipients used as bulking agents during the spray drying/granulation process of this invention are lactose, mannitol, Povidone (PVP), sucrose, sodium starch glycolate, and microcrystalline cellulose, which are incorporated in stable oral solid dosage forms of eprosartan by blending with additional excipients in desired proportions. More preferably, the excipients used as bulking agents during the spray drying/granulation process are mannitol/lactose, microcrystalline cellulose, sucrose, sodium starch glycolate and Povidone (PVP). Most preferably, the excipients used as bulking agents during the spray drying/granulation process are lactose, microcrystalline cellulose and sodium carmellose.

Preferably, the bulking agents used in the formulation are present in an amount of about 2% to about 80% on a weight for weight basis. Most preferably, the bulking agent(s) may be present at as low as about 5% to about 50% on a weight for weight basis.

The process for preparing the solid dosage forms in accordance with the present invention may be carried out using a combination of a blender/stirrer, a spray dryer or a fluid bed granulator, a comminuting mill, sieving equipment, a powder blender, a capsule filling machine or a tableting machine. Optionally, the spray-dried material may be processed using a rotogranulator to produce spherical granules, which may be polymer film coated to impart modified release properties. Tablets of the spray dried/fluid bed granules may be optionally polymer film coated to produce delayed, sustained, or targeted release dosage forms.

Thus, one embodiment provides a pharmaceutical composition, which comprises (E)-α-[2-n-butyl-1-[(4-carboxyphenyl)methyl]-1H-imidazol-5-yl]methylene-2-thiophenepropionic acid. The pharmaceutical composition is adapted for oral administration. The composition is presented as a unit dose pharmaceutical composition containing from about 410 to about 490 mg of (E)-α-[2-n-butyl-1-[(4-carboxyphenyl)methyl]-1H-imidazol-5-yl]methylene-2-thiopheneprop ionic acid, preferably about 420 to about 480 mg, more preferably about 430 to about 470 mg, still more preferably about 440 to about 460 mg, and most preferably about 450 mg. Such a composition is normally taken one time daily. The preferred unit dosage forms include tablets or capsules. The compositions may be formulated by conventional methods of admixture, such as blending, filling and compressing. Suitable pharmaceutically acceptable excipients for use in this invention include diluents, fillers, binders and disintegrants. Preferably, the composition is a dry-mix formulation as described above.

(E)-α-[2-n-Butyl-1-[(4-carboxyphenyl)methyl]-1H-imidazol-5-yl]methylene-2-thio-phenepropionic acid may be co-administered with other pharmaceutically active compounds, for example, in physical combination or by sequential administration. Conveniently, the compound of this invention and the other active compound are formulated in a pharmaceutical composition. Thus, a further embodiment relates to pharmaceutical compositions comprising (E)-α-[2-n-butyl-1-[(4-carboxyphenyl)methyl]-1H-imidazol-5-yl]-methylene-2-thiophenepropionic acid, a pharmaceutically acceptable carrier, and a second pharmaceutically active compound selected from the group consisting of: a diuretic, a calcium channel blocker, a β-adrenoceptor blocker, a renin inhibitor, and an angiotensin converting enzyme inhibitor. Examples include diuretics, particularly a thiazide diuretic, such as hydrochlorothiazide, or a loop diuretic, such as furosemide, calcium channel blockers, particularly dihydropyridine antagonists, such as nifedipine, β-adrenoceptor blockers, such as propranolol, renin inhibitors, such as enalkinen, and angiotensin converting enzyme inhibitors, such as captopril or enalapril. Preferably, the pharmaceutical composition contains about 200 to about 400 mg of (E)-α-[2-n-butyl-1-[(4-carboxyphenyl)-methyl]-1H-imidazol-5-yl]methylene-2-thiophene-propionic acid in combination with about 6.25 to about 25 mg of hydrochlorothiazide.

No unacceptable toxicological effects are expected when (E)-α-[2-n-butyl-1-[(4-carboxyphenyl)methyl]-1H-imidazol-5-yl]methylene-2-thiophenepropionic acid is administered in accordance with the present invention.

(E)-α-[2-n-Butyl-1-[(4-carboxyphenyl)methyl]-1H-imidazol-5-yl]methylene-2-thio-phenepropionic acid is useful for treating diseases in which blockade of the All receptor would be beneficial. Preferably, this compound is used, alone or in combination with said second pharmaceutically active compounds, in the treatment of hypertension, congestive heart failure and renal failure. Additionally, (E)-α-[2-n-butyl-1-[(4-carboxy-phenyl)methyl]-1H-imidazol-5-yl]methylene-2-thiophenepropionic acid is of value in the treatment of left ventricular hypertrophy regression, diabetic nephropathy, diabetic retinopathy, muscular degeneration, hemorrhagic stroke, primary and secondary prevention of infarction, prevention of atheroma progression and the regression of atheroma, prevention of restinosis after angioplasty or bypass surgery, improving cognitive function, angina, glaucoma, and CNS disorders, such as anxiety.

The following examples are illustrative of the instant invention. These examples are not intended to limit the scope of this invention as defined hereinabove and as claimed herein below.

### Example 1a

### Analytical Methods

XRPD patterns were measured on a diffractometer using monochromatic CuKα radiation (tube voltage 40 kV, tube current 40 mA) at room temperature. IR spectra were recorded on a Fourier transform IR spectrometer in attenuated total reflectance (silicon crystal) with a spectral resolution of 2 cm⁻¹ using a mercury cadmium telluride detector. Melting points were determined on a DSC apparatus as onset temperatures of the melting endotherm using 40 μL aluminum crucibles with a pierced lid. Temperature program: heating from 25° C. up to 300° C. with 10 K min⁻¹. N₂ atmosphere at a flow of 80 mL min⁻¹.

### Example 1b

### Materials

Eprosartan acid can be prepared as described in U.S. Pat. No. 5,185,351. The formulation, indicated as “commercial tablet” contains eprosartan mesylate in an amount corresponding with 600 mg eprosartan, and is commercially available. In addition to eprosartan acid, the formulation contains microcrystalline cellulose, lactose monydrate, pregelatinised starch, crospovidone and magnesium stearate in the core, and Opadry White, Hypromellose, Macrogol 400, Polysorbate 80 and Titanium dioxide in the coating.

### Example 2

### Preparation of Polymorphic Form α of Eprosartan Acid

### Example 2.1 Preparation of α Polymorphic Form of Eprosartan Acid from Acetic Acid

Eprosartan acid (50 g) was dissolved in 125 ml acetic acid by heating to 110° C. The solution was cooled to 20° C. in about 1 hour and aged at this temperature for 1 hour. The product was isolated by filtration, washed twice with 50 ml water and successively dried at 65° C. under vacuum to give 41 g eprosartan in the α polymorph. The XRPD pattern of the α polymorphic form is given in FIG. 1.

### Example 2.2 Preparation of α Polymorphic Form of Eprosartan Acid from Acetic Acid/Methanol

Eprosartan acid (100 g) was dissolved in 200 ml acetic acid by heating to 110° C. The solution was cooled to 10° C. in about 1 hour; during this cooling, starting from 70° C., 200 ml methanol was added slowly. The crystallization started at 52° C. when approximately half of the methanol was added. The resulting slurry was aged at 10° C. for 1 hour. The product was isolated by filtration, washed twice with 100 ml methanol and successively dried at 65° C. under vacuum to give 93 g eprosartan in the α polymorph.

This procedure can also be carried out using ethyl acetate, isopropanol, ethanol, acetone, acetonitrile or water instead of methanol.

### Example 2.3 Preparation of α Polymorphic Form of Eprosartan Acid from Formic Acid/Water

Eprosartan (50 g) was dissolved in 75 ml formic acid by heating to 50° C. At this temperature, 200 ml water was added in about 40 minutes to crystallize the product. The resulting slurry was cooled to 15° C. in about 30 minutes and aged at this temperature for 1 hour. The product was isolated by filtration, washed twice with 50 ml water and successively dried at 65° C. under vacuum to give 45 g eprosartan in the α polymorph.

### Example 2.4 Preparation of α Polymorphic Form of Eprosartan Acid from Ethanol/Water by Seeding

A slurry of eprosartan (106.8 g water wet, containing approximately 100 g eprosartan) in 125 ml ethanol and 73 ml water was heated to 55° C., while 61.8 g of an aqueous 32% sodium hydroxide solution was slowly added, to give a clear yellow solution having a pH of 12.9.

At 55° C., the solution was acidified with 32% hydrochloric acid until pH 6.4. At this pH, the crystallization was induced by seeding with 2 g eprosartan having the α polymorph. After aging the crystal slurry for 30 minutes, the acidification was continued to a final pH of 5.2.

The slurry was cooled to 20° C. before the product was isolated by filtration. The product was washed twice with 100 ml of a 1:1 mixture of ethanol and water and dried at 65° C. under vacuum to give 97 g eprosartan in the α (alpha) polymorph.

### Example 3

### Preparation of Polymorphic Form β of Eprosartan Acid

A slurry of eprosartan (100 g) in 125 ml ethanol and 75 ml water was heated to 55° C., while 62.6 g of an aqueous 32% sodium hydroxide solution was slowly added, to give a clear yellow solution having a pH of 12.8.

At 55° C., the solution was acidified with 37% hydrochloric acid until pH 5.2 to crystallize the product. The slurry was cooled to 20° C. and aged at this temperature for 1 hour before the product was isolated by filtration. The product was washed 3 times with 100 ml of a 1:1 mixture of ethanol and water and dried at 65° C. under vacuum to give 97 g eprosartan in the beta polymorph. The XRPD pattern of β polymorphic form is given in FIG. 2.

### Example 4

### Rearrangement of β Polymorphic Form into α Polymorphic Form of Eprosartan Acid

Mixtures of α and β polymorphs of eprosartan (6 g each) were stirred at 60° C. under the following conditions:

a) The eprosartan mixture was suspended in 15 ml ethanol, 18 ml water, 3.47 g NaCl and 0.56 ml 37% hydrochloric acid.
b) The eprosartan mixture was suspended in 15 ml ethanol, 18 ml water.
c) The eprosartan mixture was suspended in 15 ml formic acid and 40 ml water.

The mixtures were sampled at regular intervals. The sample was filtered to isolate the eprosartan, which was then washed with water and dried before analysis on polymorphism. The samples obtained from condition a) and c) showed full conversion into the α polymorph after 6 hours. The sample obtained from condition b) showed full conversion into the α polymorph after 1 week.

### Example 5

### Preparation of a Formulation Containing 420 mg Eprosartan Acid by Wet Granulation

A formulation is prepared having the composition listed in Table 1.

TABLE 1

| | | | | |
|---|---|---|---|---|
| | | | | Reference to |
| Component | % w/w | mg/700 mg* | Function | Standard |

Intra-granular Components

| | | | | |
|---|---|---|---|---|
| Eprosartan acid | 60.0 | 420.0 | Active | Internal |
| | | | | Monograph |
| Pharmatose 200M | 19.0 | 133.0 | Diluent | Ph. Eur. |
| Avicel PH102 | 8.0 | 56.0 | Compression | Ph. Eur. |
| | | | Aid | |
| Starch 1500 | 8.0 | 56.0 | Binder | Ph. Eur. |
| Ac-Di-Sol | 3.0 | 21.0 | Disintegrant | Ph. Eur. |

Extra-granular Components

| | | | | |
|---|---|---|---|---|
| Ac-Di-Sol | 1.0 | 7.0 | Disintegrant | Ph. Eur. |
| Magnesium Stearate | 1.0 | 7.0 | Lubricant | Ph. Eur. |

Film Coating

| | | | | |
|---|---|---|---|---|
| Opadry Yellow | 4.0 | 28.0 | Coating | Internal |
| 03B22291 | | | | Monograph |
| Total Tablet weight | | 728 mg | | |

*Tablet core weight (uncoated) is 700 mg. Composition is expressed based on core tablet weight

Eprosartan acid (600 g), Pharmatose 200M (190 g), Avicel PH102 (80 g), Starch 1500 (80 g), and Ac-Di-Sol (30 g) are screened (1000 μm) and dry-mixed. Purified water is added, followed by wet massing and drying. Magnesium stearate (10 g) and Ac-Di-Sol (10 g) are added and the resulting mixture is compressed into tablets. The tablets are coated with Opadry yellow O3B222291.

### Example 6

### Preparation of a Formulation Containing 420 mg Eprosartan Acid and Effersoda-12™

A formulation is prepared having the composition listed in Table 2.

TABLE 2

| | | | | |
|---|---|---|---|---|
| | | | | Reference to |
| Component | % w/w | mg/900 mg* | Function | Standard |

Intra-granular Components

| | | | | |
|---|---|---|---|---|
| Eprosartan acid | 46.7 | 420.0 | Active | Internal |
| | | | | Monograph |
| Effer-Soda12 ™ | 22.2 | 200.0 | Effervescent | Ph. Eur. |
| Pharmatose DCL21 | 26.6 | 239.5 | Filler | Ph. Eur. |
| Sodium Starch | 1.5 | 13.5 | Disintegrant | Ph. Eur. |
| Glycolate | | | | |
| Magnesium stearate | 1.0 | 9.0 | Lubricant | Ph. Eur. |

Extra-granular Components

| | | | | |
|---|---|---|---|---|
| Sodium Starch | 1.5 | 13.5 | Disintegrant | Ph. Eur. |
| Glycolate | | | | |
| Magnesium Stearate | 0.5 | 4.5 | Lubricant | Ph. Eur. |

Film Coating

| | | | | |
|---|---|---|---|---|
| Opadry II Yellow | 4.0 | 36.0 | Coating | Internal |
| 85F22122 | | | | Monograph |
| Total Tablet weight | | 936 mg | | |

*Tablet core weight (uncoated) is 900 mg. Composition is expressed based on core tablet weight

Eprosartan free acid (467 g), Effer-Soda-12™ 12 (222 g), sodium starch glycolate (15 g) and lactose monohydrate (266 g) are screened (1000μ) and blended for 10 minutes. Magnesium stearate (10 g) is added, followed by 2 minutes blending. The resulting mixture is slugged and milled twice. Magnesium stearate (5 mg) and sodium starch glycolate (15 g) are added, followed by blending and compression into tablets. The tablets are filmcoated with Opadry I 85F22122.

### Example 7

### Preparation of a Formulation Containing 450 mg Eprosartan

A formulation is prepared on the basis of the composition outlined in Table 3 below. To this end, Eprosartan Free Acid (20.809 kg), Lactose Monohydrate 200M (3.295 kg), Prosolv SMCC90 (2.775 kg), Starch 1500 Pregelatinised (2.775 kg) and Crospovidone (Polyplasdone XL10) (0.347 kg) are screened (1000 micron) and dry mixed. Purified water is added followed by wet massing, milling and drying. Magnesium Stearate LIGA MF-2-V (1.13% w/w) and Crospovidone (Polyplasdone XL10) (1.13% w/w). are added and the resulting mixture is compressed into tablets. The tablets are coated with Opadry 03B22291.

TABLE 3

| | | | | |
|---|---|---|---|---|
| | % w/w per | mg/663.8 | | Reference to |
| Component | 663.8 mg | mg | Function | Standard |

Intra granular

| | | | | |
|---|---|---|---|---|
| Eprosartan Free Acid | 67.797 | 450.0 | Active | Internal |
| | | | | Monograph |
| Lactose Monohydrate | 10.734 | 71.3 | Substrate for | Ph. Eur. |
| Pharmatose 200M | | | granulation | |
| Silicified MCC | 9.04 | 60.0 | Substrate for | Ph. Eur. |
| ProSolv ® SMCC 90 | | | granulation/ | |
| | | | compression aid | |
| Starch Pregelatinised 1500 | 9.04 | 60.0 | Binder | Ph. Eur. |
| Crospovidone | 1.13 | 7.5 | Disintegrant | Ph. Eur. |
| Polyplasdone XL-10 | | | | |
| Purified Water¹ | (42¹) | — | Solvent/Diluent | Ph. Eur. |

| | | |
|---|---|---|
| Sub Total | 97.74 | 648.8 |

Extra granular

| Crospovidone | 1.13 | 7.5 | Disintegrant | Ph. Eur. |
|---|---|---|---|---|
| Polyplasdone XL-10 | | | | |
| Magnesium Stearate | 1.13 | 7.5 | Lubricant | Ph. Eur. |
| Liga MF-2-V | | | | |
| Sub Total | 100.0 | 663.8 | | |
| Opadry 03B22291 | 3² | 19.9 | Coating Agent | Internal |
| Yellow | | | | Monograph |
| Purified Water¹ | — | (139.4) | Solvent/Diluent | |

| | | |
|---|---|---|
| Total | N/A | 690.4 |

¹Removed during the process, 42% of granulation batch size
²3% Weight gain (Coating solution = 12.5% w/w total solids)

### Example 8

### Release Profile Eprosartan from Different Formulations

Dissolution testing from different eprosartan tablets was performed using the USP Dissolution Apparatus II with a dissolution medium 0.2 M phosphate buffer at pH 7.5, a medium volume of 1000 ml with a temperature of 37±0.5° C. at a paddle speed of 50 rpm, taking 10 ml samples and measuring in a QS Flow cell with a path length of 1 mm at a wavelength of 235 nm. The 0.2 M phosphate buffer was prepared by dissolving 302.6 g of disodium hydrogen phosphate dihydrate and 40.8 g of potassium dihydrogen phosphate in 10 liters of pure water. The pH was adjusted to 7.50±0.05 with addition of either 5 M sodium hydroxide or 85% phosphoric acid.

The dissolution profiles were as follows:

TABLE 4

| | Eprosartan marketed | 420 mg tablet | 450 mg tablet |
|---|---|---|---|
| | 600 mg mesylate tablet | (Example 5) | (Example 7) |
| Time (min) | (% release) | (% release) | (% release) |
| 0 | 0 | 0 | 0 |
| 5 | | 60 | 36 |
| 10 | 84 | | |
| 15 | 92 | 100 | 99 |
| 20 | 95 | | |
| 30 | 97 | 100 | 100 |
| 45 | 98 | 100 | 99 |
| 60 | | 100 | 100 |

### Example 9

### An Open-Label, Randomized, Three-Way Cross-Over Evaluation of the Relative Bioavailability of Two 420 mg Free Acid Eprosartan Tablets in Comparison to the Marketed 600 mg Tablets of Eprosartan in Healthy Adult Male Subjects

Objectives:

Primary Objective

To assess the relative bioavailability of two experimental 420 mg eprosartan free acid tablets with the reference formulation: eprosartan 600 mg (as a mesylate) marketed tablets in healthy adult, male, human subjects under fasting conditions.

Secondary Objective

To assess the safety and tolerability of the eprosartan formulations.

Methodology:

This was a single center, open-label, balanced, randomized, single dose, cross-over, comparative oral bioavailability study in 24 healthy, adult, male subjects under fasting conditions. Each subject was to participate in three study periods separated by a washout period of at least five days. Subjects received one of the following treatments in each treatment period; Treatment A (experimental 420 mg eprosartan free acid effersoda tablet according to Example 6), Treatment B (experimental 420 mg eprosartan free acid pharmatose tablet according to Example 5) and Treatment C (600 mg eprosartan marketed tablets). Subjects were screened for their eligibility to participate in the study within 28 days of their first admission. Eligible subjects were admitted to the clinic on the day prior to dosing (Day-1) and randomized to treatment. Subjects remained in the clinical unit until Day 3 of each treatment period. There was at least 5 days between dosing in adjacent treatment periods. Subjects returned to the clinical unit for a follow-up visit 5-7 days after discharge from treatment Period 3.

Number of Subjects (Planned, Randomized and Analyzed):

24 subjects were planned, randomized and analyzed.

Main Criteria for Inclusion:

Male subjects aged 18 to 50 years, inclusive with a body mass index (BMI) between 18 to 28 kg/m², inclusive with a body weight >50 kg or <100 kg. Subjects were to be in good health as determined by vital signs, medical history, physical examination, serum biochemistry, urinalysis and hematology.

Test Product, Dose and Mode of Administration, Batch Number:

Treatment A: 420 mg eprosartan free acid effersoda oral tablet prepared in accordance with Example 6.

Treatment B: 420 mg eprosartan free acid pharmatose oral tablet, prepared in accordance with Example 5.

Duration of Treatment:

Subjects received a single dose of Treatment A, B and C in three treatment periods. There was at least 5 days between dosing in adjacent periods.

Reference Therapy, Dose and Mode of Administration, Batch Number:

Treatment C: 600 mg eprosartan marketed tablets,

Criteria for Evaluation

Pharmacokinetics:

The following pharmacokinetic (PK) parameters for eprosartan were determined: λ_{z}, AUC, AUC/D, AUC₀₋ₜ, AUC₀₋ₜ/D, Cₘₐₓ, Cₘₐₓ/D, CL/F, t_{1/2}, tₘₐₓ, V_{Z}/F and bioavailability parameters (F_{AUC} %, F_{AUC(0-t)} %, F_{Cmax} %, F_{AUC/D} %, F_{AUC(0-t)/D} %, F_{Cmax/D} %). The AUC, AUC₀₋ₜ, and Cₘₐₓ were considered as the primary PK parameters.

Statistical Methods:

Pharmacokinetics:

Plasma concentrations of eprosartan were summarized by treatment and nominal measurement time using descriptive statistics. Concentrations below the LLOQ were set to ½ LLOQ prior to calculation of descriptive statistics. Descriptive statistics were only calculated if at least ⅔ of the data were ≧LLOQ. Eprosartan pharmacokinetic parameters were summarized by treatment using descriptive statistics.

A mixed model analysis of variance (ANOVA) of the primary pharmacokinetic parameters Cₘₐₓ, AUC₀₋ₜ, and AUC was used to compare the relative bioavailability of the test formulations (experimental 420 mg free acid tablets) with the reference formulation (eprosartan 600 mg tablet). A model with fixed effect terms for period, sequence and treatment and subject within sequence as random effect was used. The pharmacokinetic parameters were log transformed before analysis.

From this ANOVA, least-squares means for each treatment, estimated treatment differences, and 90% confidence intervals for treatment differences were calculated. The log-transformed results were transformed to the original scale by exponentiation to obtain geometric least squares means, treatment ratios and their 90% confidence intervals.

Similar relative bioavailability comparisons between the test and the reference formulations were performed on dose-normalized parameters (Cₘₐₓ/D, AUC₀₋ₜ/D, and AUC/D).

Summary—Conclusions

Pharmacokinetic Results:

Samples were analyzed for concentrations of eprosartan using validated high performance liquid chromatography with tandem mass spectrometry detection (lower limit of quantitation: 1 ng/mL).

Following administration of the eprosartan effersoda tablet (Trt A, test formulation), pharmatose tablet (Trt B, test formulation) or the marketed tablet (Trt C, reference formulation), the plasma concentrations decreased in a mostly biphasic manner and were quantifiable for up to 48 hours post administration (last time point evaluated). The concentration time profiles for eprosartan for the three treatments were similar at later time points (>10 h) but there were differences in the profiles in the first 10 hours following dosing (i.e., absorption phase). The effersoda formulation showed a more rapid absorption and a higher Cₘₐₓ, while the pharmatose and the marketed formulations showed similar absorption profiles. The summary of key PK parameters for eprosartan is presented in Table 5:

Based on non-normalized eprosartan parameters, mean overall exposure (AUC and AUC₀₋ₜ) for the effersoda tablet was 7-12% higher and mean Cₘₐₓ was 34% higher compared to the marketed tablet. In the case of the pharmatose tablet, AUC and Cₘₐₓ were similar to the marketed tablet (<5% difference), but the mean AUC₀₋ₜ was 11% lower (see table 4).

Based on dose-normalized eprosartan parameters, mean overall exposure (AUC and AUC₀₋ₜ) for the effersoda tablet was 52-60% higher and mean Cₘₐₓ was 92% higher compared to the marketed tablet. Similarly, in the case of the pharmatose tablet, mean overall exposure (AUC and AUC₀₋ₜ) was 28-47% higher and mean Cₘₐₓ was 38% higher compared to the marketed tablet (see table 4).

TABLE 5
Summary of Key Non-Normalized and Dose-Normalized
Eprosartan Plasma Pharmacokinetic Parameters

| | Treatment A | | | Treatment B | | | Treatment C | | |
|---|---|---|---|---|---|---|---|---|---|
| Parameter | N | Mean (SD) | | N | Mean (SD) | | N | Mean (SD) | |
| AUC (ng · h/mL) | 18 | 10020 | (4969) | 18 | 9277 | (4644) | 19 | 7695 | (3846) |
| DN- AUC | 18 | 23.86 | (11.81) | 18 | 22.08 | (11.05) | 19 | 12.81 | (6.39) |
| AUC₀₋ₜ | 24 | 9470 | (5173) | 23 | 8139 | (4519) | 24 | 9415 | (7268) |
| (ng · h/mL) | | | | | | | | | |
| DN-AUC0-t | 24 | 22.53 | (12.3) | 23 | 19.39 | (10.78) | 24 | 15.70 | (12.13) |
| Cₘₐₓ (ng/mL) | 24 | 3425 | (1911) | 23 | 2533 | (1541) | 24 | 2652 | (1947) |
| DN-Cmax | 24 | 8.153 | (4.547) | 23 | 6.030 | (3.668) | 24 | 4.420 | (3.242) |
| tₘₐₓ (h)^{[a]} | 24 | 0.52 | (1.25-4.00) | 23 | 2.00 | (0.75-4.03) | 24 | 1.00 | (1.50-3.50) |
| T_{1/2} (h) | 18 | 10.47 | (3.04) | 18 | 15.6 | (10.85) | 19 | 16.14 | (9.11) |
| Vz/F (L) | 18 | 868.8 | (646.2) | 18 | 1323 | (1178) | 19 | 2398 | (1662) |
| CL/F (L/h) | 18 | 53.97 | (28.64) | 18 | 58.76 | (37.99) | 19 | 96.86 | (44.05) |

SD = standard deviation; DN = Dose-Normalized Treatment A: Eprosartan free acid (420 mg) effersoda tablet; Treatment B: Eprosartan free acid (420 mg) pharmatose tablet; Treatment C: Eprosartan (600 mg) marketed tablet
^{[a]}Median (range)

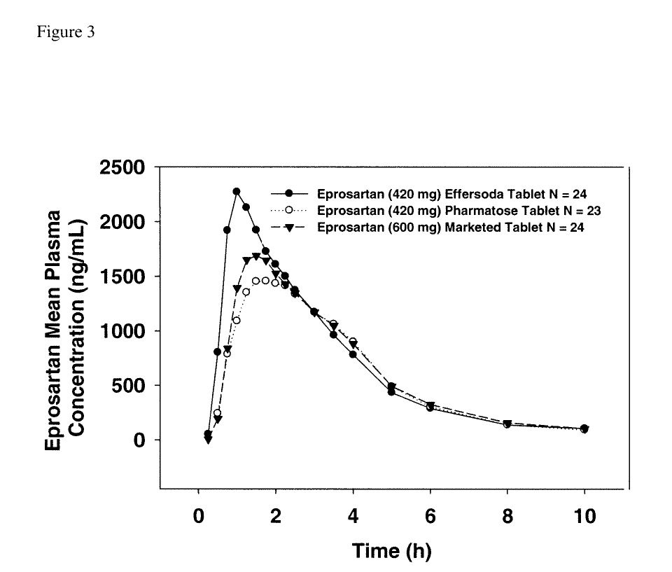

Following single dose oral administration of the eprosartan (420 mg) effersoda formulation, eprosartan (420 mg) pharmatose formulation, or the eprosartan (600 mg) marketed formulation, there was a rapid increase in the plasma concentrations of eprosartan up to approximately 0.5 to 2 hours (median tₘₐₓ), followed by a biphasic decline in plasma concentrations. Geometric mean plasma concentrations of eprosartan were quantifiable in plasma for up to 48 hours (last time point for PK evaluation) following oral administration of all the three formulations. The concentration time profiles of eprosartan for the three treatments were similar at later time points (>10 h) but there were differences in the profiles in the first 10 hours following dosing (ie, absorption phase). The geometric mean eprosartan plasma concentration time profiles for these first 10 hours are shown in FIG. 3. The effersoda formulation showed a more rapid absorption and a higher Cₘₐₓ, while the pharmatose and the marketed formulations showed similar absorption profiles.

Conclusion:

Pharmacokinetic Conclusions
The relative bioavailability of eprosartan from the effersoda formulation compared to the marketed formulation (unadjusted for dose) is 112% [90% CI: 96-127%] (AUC), 107% [90% CI: 93-123%] (AUC₀₋ₜ), and 134% [90% CI: 113-159%] (Cₘₐₓ).
The relative bioavailability of eprosartan from the pharmatose formulation compared to the marketed formulation (unadjusted for dose) is 103% [90% CI: 88-120%] (AUC), 89% [90% CI: 78-103%] (AUC₀₋ₜ), and 96% [90% CI: 81-114%] (Cₘₐₓ). The pharmatose formulation provides a similar eprosartan exposure as the marketed formulation.
When adjusted for dose differences, there is a 60% [90% CI: 38-85%] and 52% [90 % CI: 33-75%] increase in eprosartan AUC and AUC₀₋ₜ, and an approximately 92% [90% CI: 62-127%] increase in Cₘₐₓ with the effersoda formulation as compared to the marketed formulation.
When adjusted for dose differences, there is a 47% [90% CI: 26-71%] and 28% [90% CI: 11-47%] increase in eprosartan AUC and AUC₀₋ₜ, and an approximately 38% [90% CI: 16-63%] increase in Cₘₐₓ with the pharmatose formulation as compared to the marketed formulation.

As to safety, both the effersoda and pharmatose test formulations of eprosartan were well tolerated by the healthy subjects in the study.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of any aspect of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or limitation at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

1. A method of treating a disorder modulated by blocking angiotensin II (AII) receptors, wherein the disorder is selected from the group consisting of hypertension, congestive heart failure, renal failure, and combinations thereof, comprising the step of administering to a subject in need thereof a Recommended Effective Daily Dose of an eprosartan compound, wherein the eprosartan compound is eprosartan acid.

2. The method of claim 1, wherein the eprosartan acid is administered in a daily dose amount of between about 410 mg and about 490 mg.

3. The method of claim 2, wherein the daily dose amount of eprosartan acid is in an amount between about 420 mg and about 480 mg.

4. The method of claim 3, wherein the daily dose of eprosartan acid is in amount of between about 440 mg to about 460 mg.

5. The method of claim 4, wherein the daily dose of eprosartan acid is about 450 mg.

6. The method of claim 1, wherein the daily dose of eprosartan acid is 450 mg.

7. The method of claim 1, wherein the eprosartan acid is administered in a pharmaceutical formulation exhibiting a release profile of eprosartan acid, as measured in accordance with USP, of at least about 95% in about 15 minutes.

8. The method of claim 1, wherein the eprosartan acid is administered in a pharmaceutical formulation exhibiting a release profile of eprosartan acid, as measured in accordance with USP, of at least about 30% in about 5 minutes, at least about 95% in about 15 minutes, and 100% in about 30 minutes.

9. A pharmaceutical formulation comprising about 410 mg to about 490 mg of eprosartan acid.

10. The pharmaceutical formulation of claim 9, wherein the eprosartan acid is in an amount between about 420 mg and about 480 mg.

11. The pharmaceutical formulation of claim 10, wherein the eprosartan acid is in an amount between about 440 mg to about 460 mg.

12. The pharmaceutical formulation of claim 11, wherein the eprosartan acid is about 450 mg.

13. The pharmaceutical formulation of claim 9, wherein the eprosartan acid is 450 mg.

14. A pharmaceutical formulation comprising eprosartan acid in an amount between about 70% and about 80% of the calculated amount of eprosartan acid present in a comparable eprosartan mesylate formulation, wherein following administration of both formulations to human subjects, the subjects exhibit at least one of:
(a) A mean plasma Cₘₐₓ ratio between 0.8-1.25 when comparing the eprosartan acid formulation with the comparable eprosartan mesylate formulation; or
(b) A mean plasma AUC₀₋ₜ ratio between 0.8-1.25 when comparing the eprosartan acid formulation with the comparable eprosartan mesylate formulation.

15. A pharmaceutical formulation comprising about 420 mg to about 480 mg of eprosartan acid and at least one pharmaceutically acceptable excipient, wherein following administration of the composition to human subjects, the subjects exhibit at least one of:
(a) A mean plasma Cₘₐₓ ratio between 0.8-1.25 when comparing the eprosartan acid formulation with a comparable eprosartan mesylate formulation comprising 600 mg eprosartan; or
(b) A mean plasma AUC₀₋ₜ ratio between 0.8-1.25 when comparing the eprosartan acid formulation with a comparable eprosartan mesylate formulation comprising 600 mg eprosartan.

16. The pharmaceutical formulation of claims 14 or 15, wherein the eprosartan acid is in an amount between about 440 mg and 460 mg.

17. The pharmaceutical formulation of claims 16, wherein the eprosartan acid is about 450 mg.

18. The pharmaceutical formulation of claims 14 or 15, wherein the eprosartan acid is 450 mg

19. The pharmaceutical formulation of the claims 14 or 15, wherein the formulation exhibits a release profile of eprosartan acid, as measured in accordance with USP, of at least about 95% in about 15 minutes.

20. The pharmaceutical formulation of claims 14 or 15, wherein the formulation exhibits a release profile of eprosartan acid, as measured in accordance with USP, of at least about 30% in about 5 minutes, at least about 95% in about 15 minutes, and 100% in about 30 minutes.

21. The pharmaceutical formulation of the claims 14 or 15, further comprising alpha lactose monohydrate as a pharmaceutically acceptable excipient.

22. The pharmaceutical formulation of claim 21, wherein the alpha lactose monohydrate is alpha lactose 200M.

23. The pharmaceutical formulation of claim 21, further comprising microcrystalline cellulose, silicified microcrystalline cellulose, starch or cross-linked N-vinyl-2-pyrrolidone.

24. The pharmaceutical formulation of claim 23, wherein the lactose monohydrate 200M and microcrystalline cellulose, silicified microcrystalline cellulose, starch or cross-linked N-vinyl-2-pyrrolidone is present as dry-mixed granules.

25. The pharmaceutical formulation of claims 14 or 15, further comprising a diuretic compound as a second active ingredient.

26. The pharmaceutical formulation of claim 25, wherein the diuretic compound is hydrochlorothiazide.

27. The pharmaceutical formulation of claims 14 or 15, wherein the formulation is used in the treatment of a disorder modulated by blocking angiotensin II (AII) receptors, wherein the disorder is selected from the group consisting of hypertension, congestive heart failure, renal failure, and combinations thereof, by administering the formulation to a subject in need thereof.

28. A method of using eprosartan acid in a drug product that is bioequivalent with a reference drug product comprising crystalline eprosartan mesylate as the active substance, wherein the bioequivalent dose of eprosartan acid is lower than the reference dose of eprosartan mesylate, calculated on the basis of eprosartan acid.

29. A low dose eprosartan pharmaceutical formulation comprising about 410 mg to about 490 mg eprosartan acid, wherein the formulation is bioequivalent to the same formulation containing 600 mg eprosartan in the form of crystalline eprosartan mesylate.

30. The pharmaceutical formulation of claim 29, wherein the eprosartan acid in an amount between 420 mg and 480 mg.

31. A low dose eprosartan pharmaceutical formulation comprising about 210 mg to about 240 mg eprosartan acid, wherein the formulation is bioequivalent to the same formulation containing 300 mg eprosartan in the form of crystalline eprosartan mesylate.

32. The pharmaceutical formulation of claim 31, wherein the eprosartan acid is about 225 mg.

33. A pharmaceutical dosage unit comprising:
(i) 450 mg of eprosartan acid,
(ii) 71.25 mg of alpha lactose monohydrate 200M,
(iii) 60.0 mg of silicified microcrystalline cellulose,
(iv) 9.040 mg of starch,
(v) 15 mg of cross-linked N-vinyl-2-pyrrolidone, and
(vi) 7.5 mg of magnesium stearate.

34. The pharmaceutical dosage unit of claim 33, wherein the dosage unit exhibits a release profile of eprosartan acid, as measured in accordance with USP, of at least about 95% in about 15 minutes.

35. The pharmaceutical dosage unit of claim 33, wherein the dosage unit exhibits a release profile of eprosartan acid, as measured in accordance with USP, of at least about 30% in about 5 minutes, at least about 95% in about 15 minutes, and 100% in about 30 minutes.
